SEQUENCE LISTING (16 sequences)

SEQ ID NO: 1 - DNA, 30 bp, Artificial Sequence
  Description of Artificial Sequence Primer
ctaaaagagttgttgatgaggtagcaaatg

SEQ ID NO: 2 - DNA, 31 bp, Artificial Sequence
  Description of Artificial Sequence Primer
catttgctacctcatcaacaactctttttag

SEQ ID NO: 3 - DNA, 30 bp, Artificial Sequence
  Description of Artificial Sequence Primer
ccggatgctgttgatgtagaatacataaac

SEQ ID NO: 4 - DNA, 30 bp, Artificial Sequence
  Description of Artificial Sequence Primer
gtttatgtattctacatcaacagcatccgg

SEQ ID NO: 5 - DNA, 28 bp, Artificial Sequence
  Description of Artificial Sequence Primer
caaaaggcaatggattatgggataatgg

SEQ ID NO: 6 - DNA, 28 bp, Artificial Sequence
  Description of Artificial Sequence Primer
ccattatcccataatccattgccttttg

SEQ ID NO: 7 - DNA, 20 bp, Artificial Sequence
  Description of Artificial Sequence Primer
taatacgactcactataggg

SEQ ID NO: 8 - DNA, 19 bp, Artificial Sequence
  Description of Artificial Sequence Primer
gctagttattgctcagcgg

SEQ ID NO: 9 - DNA, 29 bp, Artificial Sequence
  Description of Artificial Sequence Synthetic oligonucleotide
cagttttgacacggaagtactcaaagcgg

SEQ ID NO: 10 - DNA, 29 bp, Artificial Sequence
  Description of Artificial Sequence Synthetic oligonucleotide
ccgctttgagtacttccgtgtcaaaactg

SEQ ID NO: 11 - DNA, 29 bp, Artificial Sequence
  Description of Artificial Sequence Synthetic oligonucleotide
cgccccgattctggaagaaatcgctgacg

SEQ ID NO: 12 - DNA, 29 bp, Artificial Sequence
  Description of Artificial Sequence Synthetic oligonucleotide
cgtcagcgatttcttccagaatcggggcg

SEQ ID NO: 13 - DNA, 34 bp, Artificial Sequence
  Description of Artificial Sequence Synthetic oligonucleotide
ctggatgaaatcgctgaagaatatcagggcaaac

SEQ ID NO: 14 - DNA, 34 bp, Artificial Sequence
  Description of Artificial Sequence Synthetic oligonucleotide
gtttgccctgatattcttcagcgatttcatccag

SEQ ID NO: 15 - DNA, 327 bp, Escherichia coli
  CDS (1)..(324)
agcgataaaattattcacctgactgacgacagttttgacacggatgtactcaaagcggacggggcgatcctcgtcgatttctgggcagagtggtgcggtccgtgcaaaatgatcgccccgattctggatgaaatcgctgacgaatatcagggcaaactgaccgttgcaaaactgaacatcgatcaaaaccctggcactgcgccgaaatatggcatccgtggtatcccgactctgctgctgttcaaaaacggtgaagtggcggcaaccaaagtgggtgcactgtctaaaggtcagttgaaagagttcctcgacgctaacctggcgtaa

SEQ ID NO: 16 - protein, 108 aa, Escherichia coli
SDKIIHLTDDSFDTDVLKADGAILVDFWAEWCGPCKMIAPILDEIADEYQGKLTVAKLNIDQNPGTAPKYGIRGIPTLLLFKNGEVAATKVGALSKGQLKEFLDANLA

(19) United States
(12) Patent Application Publication
(10) Pub. No.: US 2002/0115827 A1
(43) Pub. Date: Aug. 22, 2002

(54) Method of protein stabilization

(75) Inventors: Key-Sun Kim, Seoul (KR); Duck Yeon Lee, Seoul (KR)

Correspondence Address: Finnegan, Henderson, Farabow,, Garrett & Dunner, L.L.P., 1300 I Street, N.W., Washington, DC, 20005-3315, US

(73) Assignee: Korea Institute of Science and Technology

(21) Appl. No.: 09/812,777

(22) Filed: Mar. 15, 2001

(30) Foreign Application Priority Data
Oct. 27, 2000 (KR) 00-63596

(51) Int. Cl. C07K 14/435

(52) U.S. Cl. 530/350

(57) ABSTRACT

The present invention relates to a method of improving heat-stability of a protein by substituting aspartic acid, a hydrophilic amino acid present on the surface of a water-soluble protein, with glutamic acid by the site-specific mutagenesis. The substitution of aspartic acid, a hydrophilic amino acid present on most proteins, with glutamic acid, which is very similar to aspartic acid in both electrostatic feature and molecular weight, can improve the heat stability of a resulting protein without affecting its structure or the function thus enabling to extend its applications to a variety of industrial fields such as medicine, food and chemistry.

### BACKGROUND OF THE INVENTION

1. Field of the Invention

The present invention relates to a method of heat-stabilization of a protein by substituting hydrophilic amino acid residues of a water-soluble protein present on the surface of the protein with another amino acid by site-specific mutagenesis.

2. Description of the Related Art

Proteins are polymers consisting of hydrophilic and hydrophobic amino acid residues and have been widely applied to food, medicine and other biochemical industries due to the varieties of their chemical and biological functions. In industrial applications, however, the stability of a protein has been of much importance because instability of proteins during distribution or use would cause them to be easily inactivated thus incurring various unwanted problems. As a result, enormous efforts have been made to improve the stability of proteins by means of adding additives or inducing mutations. For example, mutations increasing hydrophobic interactions of a protein [Kellis et al., Nature, 333, 784-786, (1988); Karpusas et al., PNAS, 86, 8237-82411], decreasing the unfolded state entropy by introducing a disulfide bond [Matsumara et al., Nature, 342, 291-293, (1989)] or prolins in a protein [Matthews et al., PNAS, 84, 6663-6667, (1987)], or improving the helix dipole and electrostatic interactions [Nicholson et al., Nature, 336, 651-656, (1988)] have been used to improve the stability of a protein. Nevertheless, these methods can affect the structure of a given protein, and simple introductions of these methods without prior understanding of the possible effects on the protein structure may result in protein instability. Therefore, it has been in high demand to find a method to stabilize a protein without affecting the structure or the function of a given protein.

### SUMMARY OF THE INVENTION

The present invention relates to a method of heat-stabilization of a protein, and more particularly, to a method of improving heat-stability of a protein by substituting aspartic acid, a hydrophilic amino acid present on the surface of water-soluble proteins, with glutamic acid by site-specific mutagenesis. The substitution of aspartic acid, a hydrophilic amino acid present on most proteins, with glutamic acid, which is very similar to aspartic acid in both electrostatic feature and molecular weight, can improve the heat stability of a resulting protein without affecting its structure or the function thus enabling to extend its applications to a variety of industrial fields such as medicine, food and chemistry.

### BRIEF DESCRIPTION OF THE DRAWINGS

FIG. 1 shows the procedure of constructing wild type protein expression vectors of MⱼTRX and ETRX.

FIG. 2 shows a method of constructing mutant protein expression vectors of MⱼTRX and ETRX by site-specific mutagenesis by using PCR (polymerase chain reaction).

FIG. 3 is a graph that shows the heat-stabilities of both a wild type MⱼTRX and a mutant MⱼTRX, wherein three glutamic acids (Glu25, Glu36, Glu5l) were substituted with three aspartic acids. Solid line and dotted line show the excess heat capacity of a wild type MⱼTRX and a mutant MⱼTRX, respectively.

FIG. 4 is a graph that shows the heat-stabilities of both a wild type ETRX and a mutant ETRX, wherein three aspartic acids (Asp15, Asp43, Asp47) were substituted with three glutamic acids. Solid line and dotted line show the excess heat capacity of a wild type ETRX and a mutant ETRX, respectively.

FIG. 5 is a graph that compares the activity between a wild type (λ) MⱼTRX and a mutant MⱼTRX (μ).

FIG. 6 is a graph that compares the activity between a wild type (λ) ETRX and a mutant ETRX (μ).

### DETAILED DESCRIPTION OF THE INVENTION

The present invention relates to a method of heat-stabilization of a protein, and more particularly, to a method of improving heat-stability of a protein by substituting aspartic acid, a hydrophilic amino acid present on the surface of a water-soluble protein, with glutamic acid by site-specific mutagenesis. Both aspartic acid and glutamic acid are hydrophilic amino acids and thus they are usually present on the surface of most proteins and their characteristics are also very similar to each other. For example, they are both acidic amino acids having similar electrostatic properties and the only difference lies in that glutamic acid is slightly greater in molecular weight than that of aspartic acid by having one more methylene group (−CH₂). Therefore, substituting these hydrophilic amino acid residues by means of a site-specific mutagenesis can improve the heat stability of a resulting protein without affecting its structure or the function even when the precise structure of a given protein is not known.

This invention is explained in more detail based on the following Examples but they should not be construed as limiting the scope of this invention.

### EXAMPLE 1

Preparation of Methanococcus jannaschii Thioredoxin (MⱼTRX) and Escherichia coli Thioredoxin (ETRX)

(1) Preparation of Wild Type Protein Expression Vector

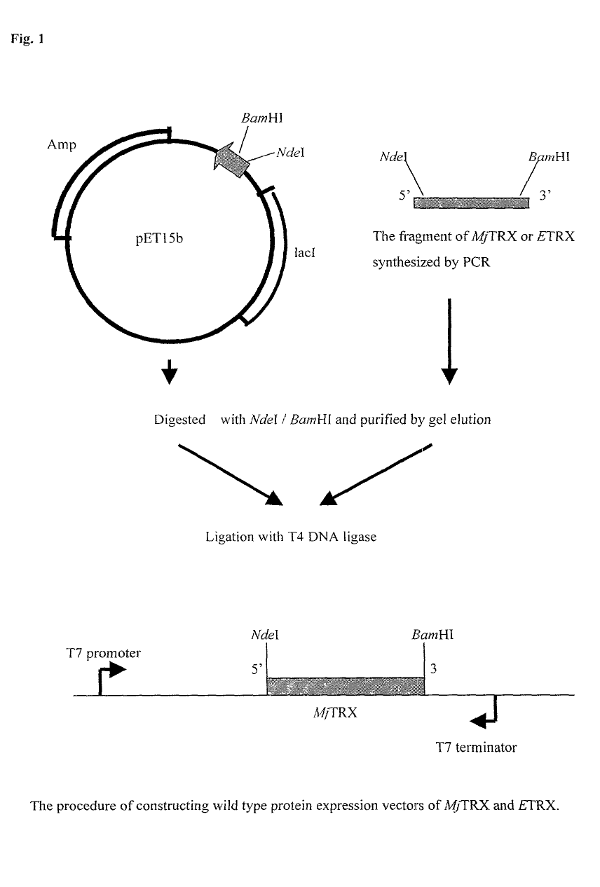

MⱼTRX and ETRX genes (SEQ ID NO: 15) were obtained by using genomic DNAs of Methanococcus jannaschii and Escilerichia coli as a DNA template, respectively, under the PCR condition of denaturing at 96° C., annealing at 58° C., and extension at 72° C. Thus obtained thioredoxin genes were then inserted into a NdeI-BamHI restriction site of pET15b (Novagen, USA) to construct an expression vector for the preparation of wild type proteins of Methanococcus jannaschii thioredoxin (MⱼTRX) and Escherichia coli thioredoxin (ETRX) (see FIG. 1).

(2) Preparation of MⱼTR Mutant Protein

MⱼTRX mutant protein expression vector was prepared by substituting each or all glutamic acids at position 25, 36, and 51 of the wild type MⱼTRX protein with aspartic acids by using the expression vector (Novagen, USA) prepared in the above step (1) with inserted MⱼTRX genes in order to prepare MⱼTRX mutant protein. Here, DNA PCR technique was employed in substituting GAA, a codon that encodes glutamic acid, with GAT, a codon that encodes aspartic acid, and the oligomers used as DNA primers are as follows (the underlined parts represent substituted codons).

| | | |
|---|---|---|
| [SEQ ID NO.1] | E25D-1: | 5′-CTAAAAGAGTTGTTGATGAGGTAGCAAATG-3′ |
| [SEQ ID NO.2] | E25D-2: | 5′-CATTTGCTACCTCATCAACAACTCTTTTTAG-3′ |
| [SEQ ID NO.3] | E36D-1: | 5′-CCGGATGCTGTTGATGTAGAATACATAAAC-3′ |
| [SEQ ID NO.4] | E36D-2: | 5′-GTTTATGTATTCTACATCAACAGCATCCGG-3′ |
| [SEQ ID NO.5] | E51D-1: | 5′-CAAAAGGCAATGGATTATGGGATAATGG-3′ |
| [SEQ ID NO.6] | E51D-2: | 5′-CCATTATCCCATAATCCATTGCCTTTTG-3′ |
| [SEQ ID NO.7] | T7 Promoter: | 5′-TAATACGACTCACTATAGGG-3′ |
| [SEQ ID NO.8] | T7 Terminator Code: | 5′-GCTAGTTATTGCTCAGCGG-3′ |

The reaction solution contains 0.1 μM of dNTP mixture, about 20 ng of pET15b wild type MⱼTRX plasmid, 1.5 unit of pfu-DNA polymrase (Stratagene, USA) and a buffer solution to make the total reaction volume to 50 μL.

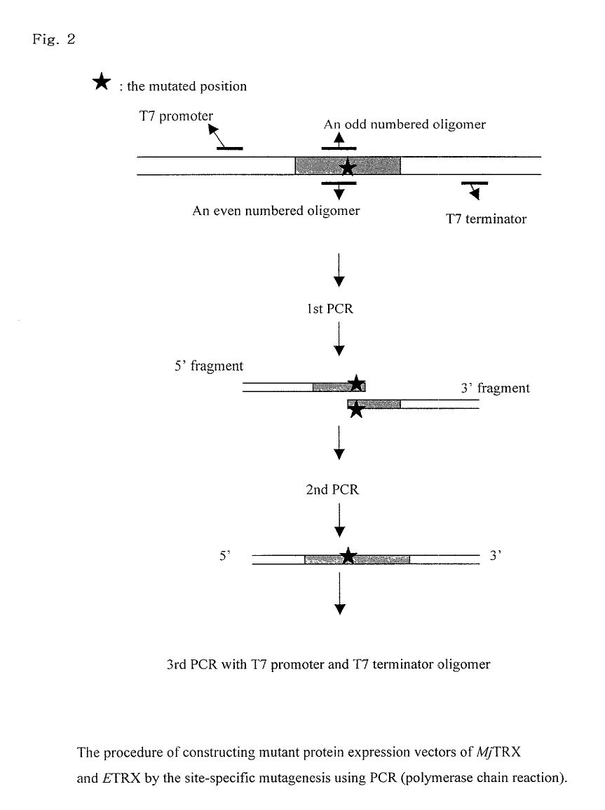

First, PCR was performed for a reaction solution containing T7 promoter oligomers and oligomers from even-numbered SEQ ID NOs, as well as for another reaction solution containing T7 terminator oligomers and oligomers from odd-numbered SEQ ID NOs under the PCR condition of 60 sec of denaturing at 96° C., 30 sec of annealing at 60° C., 50 sec of extension at 72° C. which were repeated for 30 cycles. From these PCR reactions, amplified 5′- and 3′-DNA fragments with substituted aspartic acid were obtained. Amplified DNA fragments were separated and then placed for the second round of PCR reaction. Here, only the amplified DNA fragments were added into the above reaction solution (without 20 ng of pET15b wild type MⱼTRX plasmid) with odd-numbered and even-numbered SEQ ID oligomers and PCR was performed for 10 cycles. Then, the third PCR was performed for 30 cycles under the same condition after adding T7 promoter and T7 terminator in the resulting reaction mixture of the second PCR. The mutant MⱼTRX fragments obtained from the third PCR reaction were inserted into a NdeI-BamHI restriction site of pET15b (Novagen, USA) to prepare the desired protein expression vector (see FIG. 2).

(3) Preparation of ETRX Mutant Protein

ETRX mutant protein expression vector was prepared to produce ETRX mutant protein by substituting aspartic acids at position 15, 43, and 47, with glutamic acids by using the expression vector having ETRX gene. The DNA oligomers used are shown below and the method of preparation is the same as in the preparation of MJTRX mutant proteins.

| | | |
|---|---|---|
| [SEQ 9] | D15E-1: | 5′-CAGTTTTGACACGGAAGTACTCAAAGCGG-3′ |
| [SEQ 10] | D15E-2: | 5′-CCGCTTTGAGTACTTCCGTGTCAAAACTG-3′ |
| [SEQ 11] | D43E-1: | 5′-CGCCCCGATTCTGGAAGAAATCGCTGACG-3′ |
| [SEQ 12] | D43E-2: | 5′-CGTCAGCGATTTCTTCCAGAATCGGGGCG-3′ |
| [SEQ 13] | D47E-1: | 5′-CTGGATGAAATCGCTGAAGAATATCAGGGCAAAC-3′ |
| [SEQ 14] | D47E-2: | 5′-GTTTGCCCTGATATTCTTCAGCGATTTCATCCAG-3′ |

All the mutant DNA sequences were confirmed by using an auto sequencer ABI373-DNA (Perkin Elmer, USA).

### EXAMPLE 2

Production and Separation of a Protein

The expression vectors obtained in the Example 1 were transformed into E. coli BL21(DE3). The transformed E. coli BL21(DE3) were cultured at 37° C. in LB broth containing 100 μg/mL of ampicillin until they reached an exponential growth phase (OD₆₀₀≡1.0), and then added with 0.4 mM isopropyl β-D-thiogalactopyranoside (IPTG) for the expression of wild type and mutant proteins of MⱼTRX and ETRX. Thus obtained proteins were then separated by using a nickel nitrilotriacetic acid-agarose (Ni-NTA) affinity column (Qiagen, USA) and a Superdex G75 (Pharmacia, USA) gel filtration column, and both wild type and mutant MjTRX and ETRX proteins having at least 95% purity were obtained about 10 mg per liter culture broth [Lee et al. (2000), Biochemistry, 39, 6652-6659].

### Experimental Example 1

Comparison of Thermodynamic Stability of Proteins

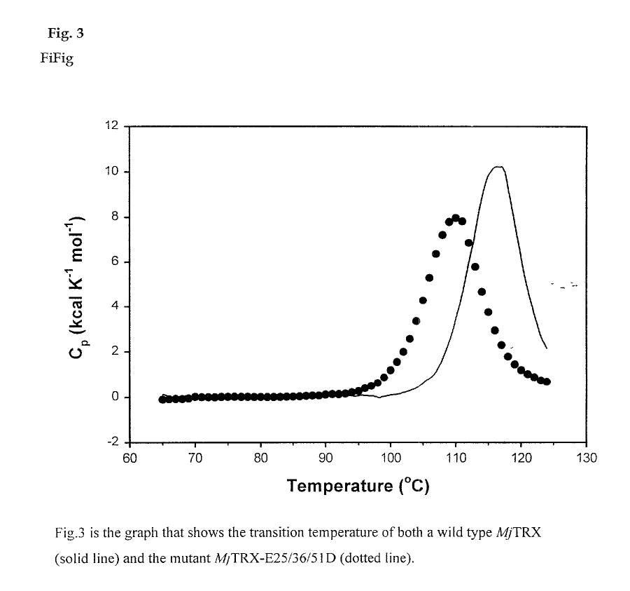

About 1 mg/mL of proteins obtained in the above Example 2 were placed in 50 mM potassium phosphate buffer solution of pH 6.5, and the excess heat capacity was obtained using a differential scanning calorimetry with the heating rate of 1° C./min. The results showed that the transition temperature (Tₘ) of the wild type MⱼTRX was 116° C. while that of the mutant MⱼTRX wherein three glutamic acids were all replaced with aspartic acids was 109° C. This is shown in FIG. 3, wherein the solid line represents a wild type MⱼTRX while the dotted line indicates a mutant MⱼTRX, and the Tₘ of the mutant MⱼTRX decreased by 2° C. per each substitution of glutamic acid with aspartic acid (Table 1).

TABLE 1

| | MjTRX | | | |
|---|---|---|---|---|
| Protein | wild type | E25D | E25/36D | E25/36/51D |
| Transition | 116.1 ± 0.3 | 114.4 ± 0.4 | 111.9 ± 0.3 | 109.4 ± 0.5 |
| Temperature | | | | |

The above table 1 is the summary of each transition temperature of wild type and mutant MjTRX.

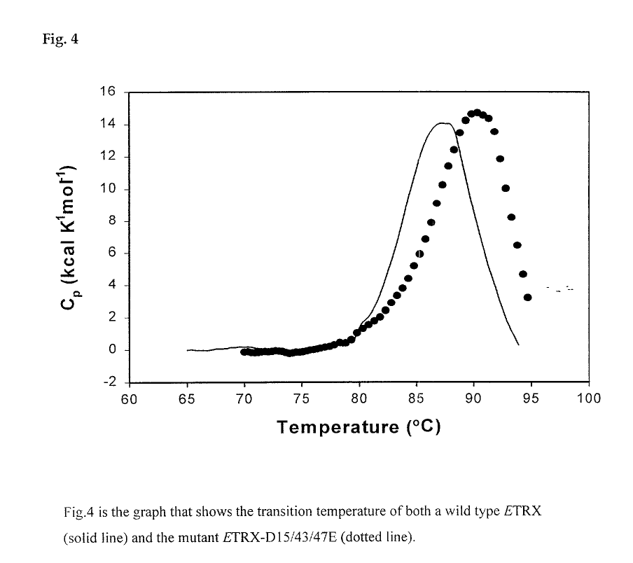

In FIG. 4, the solid line represents a wild type ETRX while the dotted line indicates a mutant ETRX, and the Tₘ of the mutant ETRX increased by 2° C. per each substitution of aspartic acid with glutamic acid; i.e., the Tₘ of the mutant ETRX, wherein three aspartic acids were replaced by glutamic acids, was raised to 92° C. from the 86° C. of the wild type ETRX, thus showing a total increase of 6° C. in Tₘ (Table 2).

TABLE 2

| | ETRX | | | |
|---|---|---|---|---|
| Protein | wild type | D15E | D43/47E | D15/43/47E |
| Transition | 86.4 ± 0.2 | 87.5 ± 0.1 | 90.4 ± 0.1 | 91.8 ± 0.2 |
| Temperature | | | | |

The above table 2 is the summary of each transition temperature of wild type and mutant ETRX.

### Experimental Example 2

The Comparison of Protein Activities

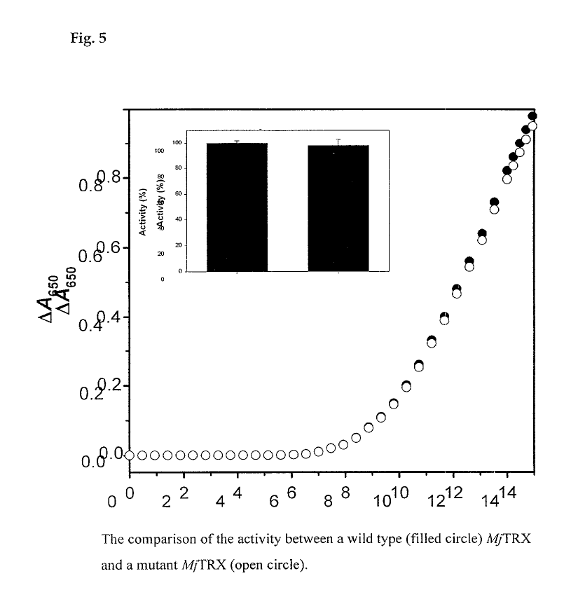

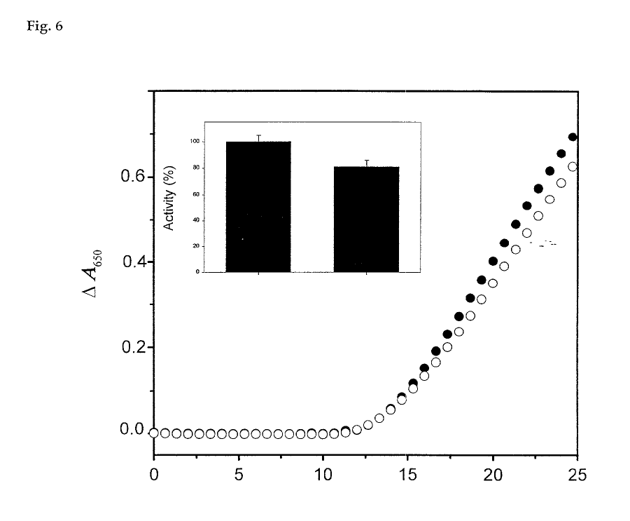

Insulin-reduction activity assay was used to measure the activities of MⱼTRX and ETRX for both a wild type and a mutant, which were produced in the Example 2. The protein concentration used in this assay was 2 μM. The degree of insulin reduction with time was measured at 650 nm and the results are shown in FIGS. 5 and 6. FIG. 5 shows the results of the assay for the activities of MⱼTRX while FIG. 6 shows that of ETRX. From the FIG. 5 it is shown that there is little difference in protein activity between a wild type and a mutant for MⱼTRX (FIG. 5) while there is a slight difference of about 7% noticed for ETRX, which in fact does not appear to be of much significance.

As described above, the present invention relates to a method of improving heat-stability of water-soluble proteins. According to the present invention, substitution of aspartic acid, a hydrophilic amino acid present on most proteins, with glutamic acid, which is very similar to aspartic acid in both electrostatic feature and molecular weight, can improve the heat stability of a resulting protein, even when its precise structure is not known, without affecting its structure or the function thus enabling to extend its applications to a variety of industrial fields such as medicine, food and chemistry.

What is claimed is:

1. A method of heat-stabilization of a protein wherein said method comprises a substitution of aspartic acid, a hydrophilic amino acid present on the surface of a water-soluble protein, with glutamic acid.
